(19) United States
(12) Patent Application Publication
(10) Pub. No.: US 2006/0047209 A1
(43) Pub. Date: Mar. 2, 2006

(54) Prosthetic heart function evaluation method and apparatus

(76) Inventor: Robert Allan Phillips, New South Wales (AU)

Correspondence Address: MERCHANT & GOULD PC, P.O. BOX 2903, MINNEAPOLIS, MN, 55402-0903, US

(21) Appl. No.: 10/520,096

(22) PCT Filed: Jun. 26, 2003
(86) PCT No.: PCT/AU03/00809

§ 371 Date: Jan. 3, 2005

(30) Foreign Application Priority Data
Jul. 3, 2002 (AU) PS 3352

(51) Int. Cl. A61B 5/02; A61N 1/362

(52) U.S. Cl. 600/504; 600/17

(57) ABSTRACT

A method of monitoring or serial measurement of the operation of a prosthetic assist device (12), the method comprising the steps of: (a) utilising a non-invasive device (21) to monitor or serially measure directly the blood flow through the by-passed heart ventricle (5); (b) separately monitoring or serially measuring the blood flow through the prosthetic assist device (12); (c) combining said two measurements to determine an overall cardiac output and a native to prosthetic flow ratio.

### FIELD OF THE INVENTION

The present invention relates to the field of monitoring of prosthetic heart devices and in particular, it discloses a form of CW Doppler monitoring of such devices.

### BACKGROUND OF THE INVENTION

The malfunctioning of the heart muscle is a major killer in society. Various devices have been proposed for assisting the operations of the cardiac function and stimulating the heart's natural pumping action. These heart assist devices come in many forms from full heart replacement to minor forms of assist, where small pumps connected to the ventricle are mounted outside the heart, and connecting flow from the ventricle to the aorta, by-passing the ventricle and systemic aortic valve.

For a survey of the field of heart (ventricular) assist devices, reference is made to:
United States Patent Application U.S. 2003/0092961 to Korakianitis publish May 15, 2003 and in particular to the references described in the Description of Related Art portion of the Application;
United States Patent Application U.S. 2003/0088147 to Boiling et al and U.S. Pat. No. 6,428,464 to Boiling and to the references referred to therein; and
U.S. Pat. No. 6,050,932 to Franchi and U.S. Pat. No. 5,267,940 to Muller.

In FIG. 1, there is shown a perspective view partly in section of the human heart. The heart can be divided into four chambers including the right atrium 2, the right ventricle 3, the left atrium 4 and the left ventricle 5. Between the chambers 2, 3 is the tricuspid valve 6. At the exit of the right ventricle is the pulmonary valve 7. Between the two chambers 4, 5 is the mitral valve 8. At the exit to the left ventricle is the aortic valve 9.

Many different prosthetic devices exist, including complete heart replacement. For example, one form of prosthetic device discussed in U.S. Pat. No. 6,428,464 amongst others bypasses the native left ventricular circulation by redirecting flow from the left ventricle 5 to the thoracic aorta 11. These prosthetic devices are designed to offload the ventricle 5 by redirecting flow from the ventricle mechanically, and encouraging the native ventricle to increase its function under decreased load. Unfortunately, in practice it is difficult to determine the effectiveness of operation of such devices. It would therefore be desirable to provide for accurate flow monitoring of heart assist devices for the analysis of the proper functioning of such devices.

### SUMMARY OF THE INVENTION

It is an object of the present invention to provide for an improved form of monitoring of the flow through the heart in conjunction with a prosthetic assist device.

In accordance with a first aspect of the present invention, there is provided a method of monitoring or serial measurement of the operation of a prosthetic assist device, the method comprising the steps of: (a) utilising a non-invasive device to monitor or serially measure directly the blood flow through the by-passed heart ventricle; (b) separately monitoring or serially measuring the blood flow through the prosthetic assist device; (c) combining said two measurements to determine an overall cardiac output and a native to prosthetic flow ratio.

Preferably, the non-invasive device monitoring comprises continuous wave Doppler flow monitoring of the heart. The monitoring can occur from a transducer placed over the parasternal acoustic access and can be repeated under a number of different operational conditions for a patient including walking and/or running. Ideally, the method is repeated under a number of different physiological and pharmalogical conditions for a patient.

### BRIEF DESCRIPTION OF THE DRAWINGS

The preferred embodiments of the present invention will now be described with reference to the accompanying drawings in which:

FIG. 1 is a perspective view partly in section of the human heart;

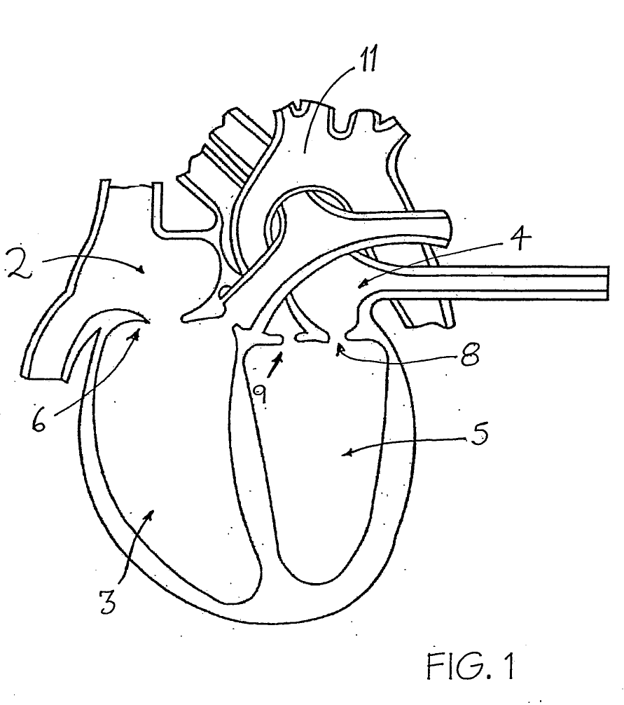

FIG. 2 illustrates schematically the operation of a prosthetic assist device;

FIG. 3 illustrates utilisation of a CW transducer device for monitoring heart function;

FIG. 4 illustrates the transducer device of FIG. 3;

FIG. 5 illustrates one form of output of the transducer device; and

FIG. 6 illustrates the various portions output in the arrangement of FIG. 5.

### DESCRIPTION OF THE PREFERRED AND OTHER EMBODIMENTS

In the preferred embodiment, accurate measurement of total ventricular function is provided by continuous wave Doppler (CW Doppler) measurement of the native ventricular function in addition to accurate flow measurement through the mechanical device.

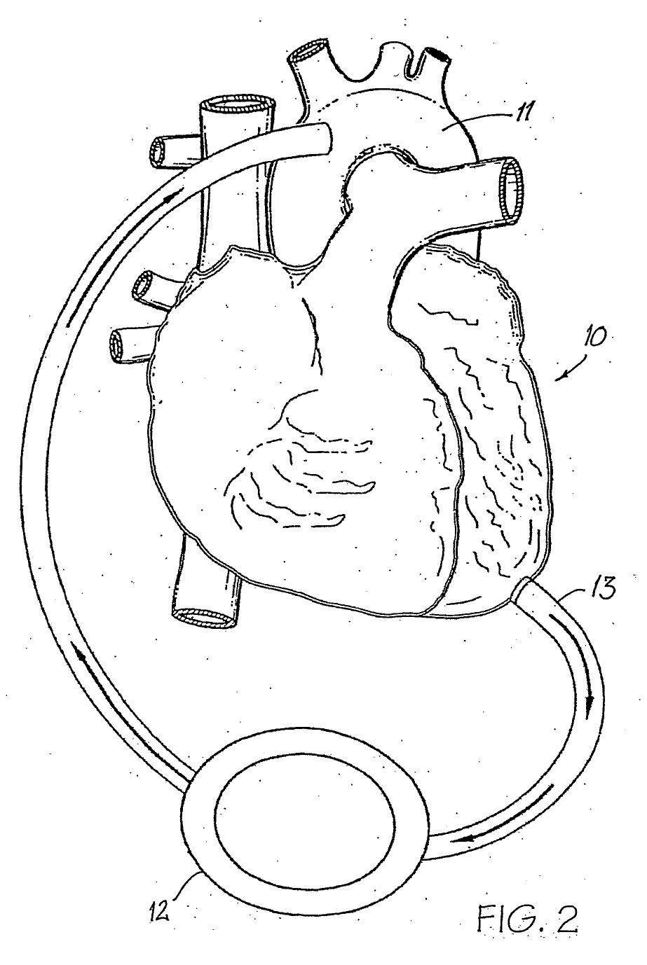

Turning initially to FIG. 2, there is illustrated a schematic of the human heart 10 which includes the aorta 11 and an additional mechanical assist device 12 which has a shunted ventricle input 13 and an aorta output 14. Whilst the flow through the mechanical assist device 12 can often be accurately measured, the flow out of the heart 10 via aorta 11 is less accurately measurable.

In the preferred embodiment, techniques of CW measurement are utilised so as to provide for flow measurements produced by the heart.

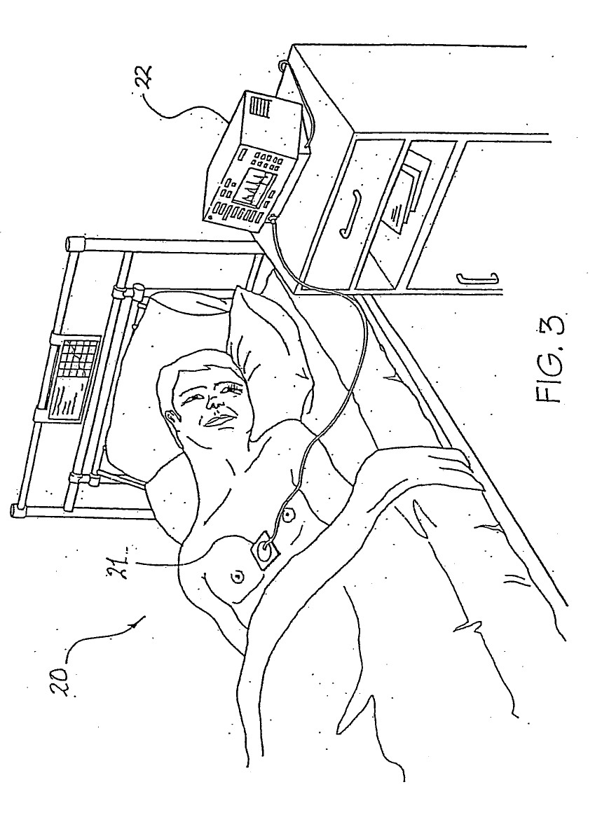

Turning to FIG. 3, there is shown a patient 20 having a non-invasive sensor 21 attached to the body. The sensor 21 comprises a CW transducer device which is interconnected to a base station unit 22.

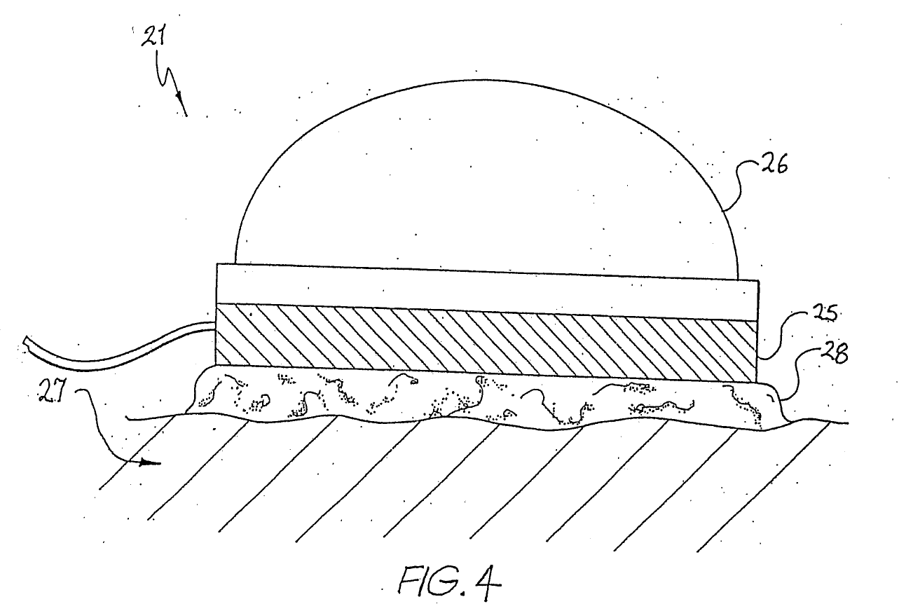

FIG. 4 shows an example of the first sensor transducer actuator 11 for attachment to the skin surface. CW Doppler measurements are utilised to monitor the blood flow through the heart. CW Doppler is a non-invasive technique in which ultrasonic signals from transducer elements are directed into a blood carrying vessel of a patient. Doppler shifts in the reflected signal provide an indication of the rate of blood flow. In FIG. 2, a transducer element 21 includes an ultrasonic transducer 25 attached to a positioning device 26 which can be used to initially set the position of the transducer. Between the transducer 25 and a patient's skin 27 is placed a gel coupling layer 28 for coupling the ultrasonic transducer vibrations to the skin 27. The principles of CW Doppler flow measurement are known are set out in more detail in Patent Cooperation Treaty (PCT) publication number WO 99/66835 entitled. “Ultrasonic Cardiac Output Monitor” assigned to the present assignee, the contents of which are incorporated herein by cross-reference. This citation describes in more detail an ultrasonic transducer device suitable for measuring blood flow within the heart using the CW Doppler method. The teaching of the above application have been embodied in an operational form in an Ultrasonic Cardiac Output Monitor available from the assignee of the present application.

In the embodiment shown in FIG. 3, the transducer elements are placed on the patient to obtain intra-cardiac or aortic signals, for example through a suprasternal notch.

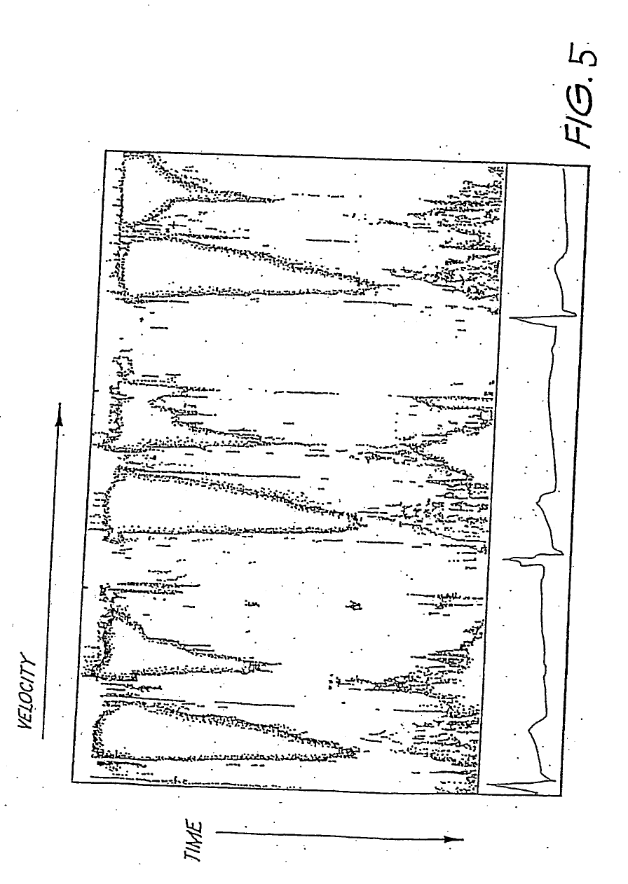

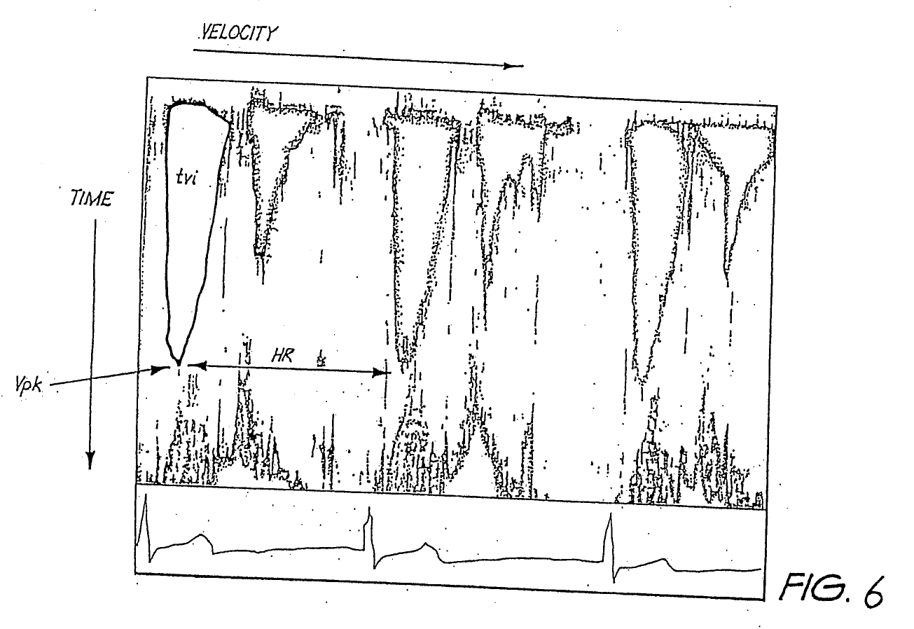

The CW method detects the velocity of individual blood cells by measuring the frequency change of a reflected ultrasound beam and displaying this as a velocity time flow profile, an example of which is shown in FIG. 5. The transducer output forms an input to the processor unit 22 of FIG. 3. From the velocity time flow profile, the processor can calculate the velocity time integral (vti) and other relevant information such as heart rate (HR), each of which is illustrated in FIG. 6. The information can be derived from FIG. 6 by appropriate image processing of the CW output image.

The total systemic output, or the total volume of blood directed into the systemic vessels, is the sum of the native plus the prosthetic output. The ratio of the two flows can be denoted a native to prosthetic flow index and can be utilised to determine appropriate levels for setting the prosthetic output of the prosthetic device 12 of FIG. 2.

Preferably, after fitting the heart assist device, the performance of the device relative to heart rate measurements are conducted under a number of different conditions.

Through measuring the device performance for a wide range of patients under a wide range of different conditions, an extensive database can be constructed of optimal performance conditions for utilisation of the heart assist device. In this way, the heart assist device can be tuned for sophisticated operation.

Further, the utilisation of the index and extensive measurements allows for optimisation of pharmacotherapy. The monitoring device thereby provides the ability to accurately monitor output flow from the heart function with potential therapeutic benefits.

Further, where the heart assist device does not include it own flow measurement capabilities, its input or output can be separately examined by the ultrasound transducer device so as to determine the assist devices output level.

It will be understood that the invention disclosed and defined herein extends to all alternative combinations of two or more of the individual features mentioned or evident from the text or drawings. All of these different combinations constitute various alternative aspects of the invention.

The foregoing describes the embodiments of the present invention and modifications, obvious to those skilled in the art can be made thereto, without departing from the scope of the present invention.

The claims defining the invention are as follows:

1. A method of monitoring the operation of a prothetic assist device, the method comprising the steps of:
(a) utilising a non invasive device to monitor or serially measure directly the blood flow through at least one heart ventricle;
(b) separately monitoring the blood flow through the prothetic assist device;
(c) combining said two measurements to determine an overall native to prosthetic flow index.

2. A method as claimed in claim 1 wherein said non invasive device monitoring or serial measurement comprises continuous wave Doppler flow monitoring of the heart.

3. A method as claimed in claim 1 wherein the heart is monitored or serially measured from a transducer placed adjacent the suprasternal notch.

4. A method as claimed in claim 1 wherein said method is repeated under a number of different operational conditions for a patient including walking and/or running.

5. A method as claimed in claim 1 wherein said method is repeated under a number of different pharmalogical conditions for a patient.
